(19) United States
(12) Patent Application Publication
(10) Pub. No.: US 2021/0257064 A1
(43) Pub. Date: Aug. 19, 2021

(54) METHOD, APPARATUS, ELECTRONIC DEVICE AND INTERACTIVE METHOD FOR CORRECTING MEASURED DATA, COMPUTER READABLE MEDIUM AND MEASURING DEVICE

(71) Applicants: BEIJING BOE HEALTH TECHNOLOGY CO., LTD., Beijing (CN); BOE TECHNOLOGY GROUP CO., LTD., Beijing (CN)

(72) Inventors: Xinxin LIU, Beijing (CN); Hongliang WANG, Beijing (CN)

(21) Appl. No.: 16/959,501

(22) PCT Filed: Feb. 28, 2020
(86) PCT No.: PCT/CN2020/077157

§ 371 Date: Jul. 1, 2020

(30) Foreign Application Priority Data
Apr. 28, 2019 (CN) 201910351257.4

(51) Int. Cl. G16H 10/60; G16H 20/60; G06F 16/215

(52) U.S. Cl. CPC G16H 10/60; G06F 16/215; G16H 20/60

(57) ABSTRACT

Disclosed are method, apparatus, electronic device and interactive method for correcting measured data, a computer readable medium and a measuring device. The method for correcting measured data includes acquiring at least one correcting factor; for each correcting factor of the at least one correcting factor, determining an additional attribute of the measured data corresponding to the correcting factor, and determining a correcting parameter of the correcting factor on the basis of the additional attribute; and carrying out correction on the measured data according to the correcting parameter of the at least one correcting factor.

The present application claims priority of Chinese Patent Application No. 201910351257.4 filed on Apr. 28, 2019, the disclosure of which is incorporated herein by reference in its entirety as part of the present application.

### TECHNICAL FIELD

The present disclosure relates to a field of data processing, and particularly, to a method, apparatus, electronic device and interactive method for correcting measured data, a computer readable medium and a measuring device.

### BACKGROUND

Users can utilize various measuring devices to carry out data measurement. It should be understood that in the data measuring process, measured data may be not accurate enough due to various influence factors.

### SUMMARY

According to an aspect of the present disclosure, it is provided a method for correcting measured data, comprising: acquiring at least one correcting factor; for each correcting factor of the at least one correcting factor, determining an additional attribute of the measured data corresponding to the correcting factor, and determining a correcting parameter of the correcting factor on the basis of the additional attribute; and carrying out correction on the measured data according to the correcting parameter of the at least one correcting factor.

In some embodiments, the additional attribute is at least one of user input information and pre-acquired statistical information.

In some embodiments, the measured data is weight data of a user, and the at least one correcting factor includes at least one of a wearing condition, a handheld condition and a dietary condition.

In some embodiments, when the correcting factor is the wearing condition, the additional attribute of the measured data corresponding to the wearing condition includes at least one of date information, a weather condition, a geographic position and physiological data of the user.

In some embodiments, the physiological data of the user includes at least one of height data, body shape data and gender data of the user.

In some embodiments, when the correcting factor is the handheld condition, the additional attribute of the measured data corresponding to the handheld condition includes device information of a handheld device for receiving the measured data.

In some embodiments, when the correcting factor is the dietary condition, the additional attribute of the measured data corresponding to the dietary condition includes at least one of time information, geographic position and gender information of the user.

In some embodiments, acquiring at least one correcting factor includes: in response to a user input, selecting at least one from a plurality of predefined correcting factors as the correcting factor of the measured data.

In some embodiments, the method further comprises: determining display information of an icon for indicating each correcting factor according to priority information of the at least one correcting factor.

According to another aspect of the present disclosure, it is provided an interactive method for correcting measured data, comprising: receiving the measured data from a measuring device; displaying the measured data and at least one correcting factor; determining a correcting factor to be used for carrying out correction from at least one displayed correcting factor, in response to input data of a user; determining an additional attribute corresponding to the correcting factor to be used for carrying out correction, and determining a correcting parameter on the basis of the additional attribute; carrying out correction on the measured data according to the correcting parameter; and displaying the corrected measured data.

In some embodiments, the displaying at least one correcting factor is determined on the basis of a priority of the correcting factor.

In some embodiments, the displaying at least one correcting factor includes: displaying the at least one correcting factor in a form of an icon or a list.

In some embodiments, the interactive method further comprises: changing a display mode of the at least one correcting factor, after displaying the corrected measured data; and updating display of the corrected measured data, in response to the user canceling the input data.

According to another aspect of the present disclosure, it is provided an apparatus for correcting measured data, comprising: an acquiring unit, configured to acquire at least one correcting factor; a parameter determining unit, configured to, for each correcting factor in the at least one correcting factor, determine an additional attribute of the measured data corresponding to the correcting factor and determine a correcting parameter of the correcting factor on the basis of the additional attribute; and a correcting unit, configured to correct the measured data according to the correcting parameter of the at least one correcting factor.

In some embodiments, the additional attribute is at least one of user input information and pre-acquired statistical information.

In some embodiments, the measured data is weight data of a user, and the at least one correcting factor includes at least one of a wearing condition, a handheld condition and a dietary condition.

According to another aspect of the present disclosure, it is provided an electronic device for correcting measured data, comprising a memory and a processor, wherein instructions are stored in the memory, and when the instructions are executed by utilizing the processor, the processor executes the above mentioned method for correcting the measured data, or executes the above mentioned interactive method for correcting the measured data.

According to another aspect of the present disclosure, it is provided a computer readable storage medium, stored thereon instructions, wherein when the instructions are executed by a processor, the processor executes the above mentioned method for correcting the measured data, or executing the above mentioned interactive method for correcting the measured data.

According to another aspect of the present disclosure, it is provided a measuring device, comprising the above mentioned electronic device for correcting the measured data; or being communicatively connected with the above mentioned electronic device for correcting the measured data.

In some embodiments, the measuring device is a weight or body fat measuring apparatus, and the electronic device for correcting the measured data is a cellphone or a tablet personal computer.

### BRIEF DESCRIPTION OF THE DRAWINGS

In order to clearly illustrate the technical solution of the embodiments of the disclosure, the drawings of description of the embodiments will be briefly described in the following. It is obvious that the described drawings are only related to some embodiments of the disclosure, and those skilled in the art also can obtain other drawings, without any inventive work, according to the drawings. The drawings below are not intentionally proportionally scaled and drawn according to an actual size, but focus on showing the substance of the present disclosure.

FIG. 1 shows an exemplary scene diagram of a data measuring system according to the present disclosure;

FIG. 2A shows a schematic flow chart of a method for correcting measured data according to an embodiment of the present disclosure;

FIG. 2B shows a schematic flow chart of an interactive method for correcting measured data according to an embodiment of the present disclosure;

FIG. 3A shows an exemplary schematic diagram of a graphical user interface according to an embodiment of the present disclosure;

FIG. 3B shows another exemplary schematic diagram of a graphical user interface according to an embodiment of the present disclosure;

FIG. 3C shows yet another exemplary schematic diagram of a graphical user interface according to an embodiment of the present disclosure;

FIG. 4 shows a schematic diagram of an apparatus for correcting measured data according to an embodiment of the present disclosure; and

FIG. 5 shows a diagram of an architecture of a computing device according to an embodiment of the present disclosure.

### DETAILED DESCRIPTION

In order to make objects, technical solutions and advantages of the embodiments of the disclosure apparent, the technical solutions of the embodiment of the disclosure will be described in a clearly and fully understandable way in connection with the drawings related to the embodiments of the disclosure. It is obvious that the described embodiments are just a part but not all of the embodiments of the disclosure. Based on the described embodiments herein, those skilled in the art can obtain other embodiment(s), without any inventive work, which should be within the scope of the disclosure.

Unless otherwise defined, the technical terms or scientific terms here should be of general meaning as understood by those ordinarily skilled in the art. In the present disclosure, words such as “first”, “second” and the like do not denote any order, quantity, or importance, but rather are used for distinguishing different components. Similarly, words such as “include” or “comprise” and the like denote that elements or objects appearing before the words of “include” or “comprise” cover the elements or the objects enumerated after the words of “include” or “comprise” or equivalents thereof, not exclusive of other elements or objects. Words such as “connected” or “connecting” and the like are not limited to physical or mechanical connections, but may include electrical connection or signal connection, either direct or indirect.

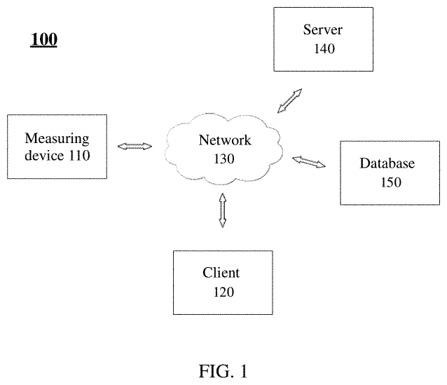

FIG. 1 shows an exemplary scene diagram of a data measuring system according to the present disclosure. As shown in FIG. 1, a system 100 may include at least one measuring device 110, a client 120, a network 130, at least one server 140 and at least one database 150.

The measuring device 110 may be a device for acquiring measured data. For example, when weight data or body fat data is to be measured, the measuring device may be a scale, e.g., a weight scale, a body fat scale and the like. When distance data is to be measured, the measuring device may be a distance meter, e.g., a ruler, a laser distance meter and the like. When time data is to be measured, the measuring device may be a second chronograph and the like. The solution does not limit the property of the measured data, and those skilled in the art should understand that the technical solution implemented according to the principle of the present disclosure can be used for the process of acquiring any measured data and for example, measuring a height, a blood pressure and the like of a human body.

The client 120 may be used for recording and/or executing correction on the measured data. In some embodiments, the client 120 may be any electronic device capable of executing data processing, such as a computer, a cellphone and the like. It should be understood that the client may be any other type of electronic device, including, but not limited to, a laptop, a tablet personal computer, a smart home device, a wearable device and the like. The client 120 provided by the present disclosure can be used for receiving the measured data from the measuring device 110 and correcting the received measured data.

In some embodiments, the received measured data may be corrected by utilizing a processing unit of the client 120. For example, the client 120 may execute the method for correcting the measured data, as provided by the present disclosure, by utilizing an algorithm and data which are stored in a built-in memory. In some implementations, the client 120 may execute correction on the measured data by utilizing a built-in application. In other implementations, the client 120 may execute correction on the measured data by calling an external application. For example, the measured data can be sent to the server 140 by the network 130, and correction on the measured data is executed by utilizing a processing unit of the server 140.

In some embodiments, the measuring device 110 and the client 120 may be integrated into the same device. For example, a device for measuring a weight and body fat, a chip with a data processing function and a display device can be integrated on a smart body fat scale, so that a user can carry out operation on the intelligent body fat scale to simultaneously measure and record weight and/or body fat data. For another example, the cellphone can be used as a second chronograph for measuring time, and meanwhile, used as the client 120 for recording and correcting measured time. In other embodiments, the measuring device 110 and the client 120 may also be implemented as separate devices. The measuring device 110 and the client 120 which are respectively independent can be connected with each other by the network 130. For example, in a case that the measuring device 110 is a smart body fat scale and the client 120 is a smartphone, information of the smart body fat scale can be registered on the smartphone, so that measured data of the smart body fat scale is recorded on the smartphone.

The network 130 may be a single network, or a combination of a plurality of different networks. For example, the network 130 may include but not limited to, one or a combination of several of a local area network, a wide area network, a public network, a private network and the like.

Wherein, an implementation of connection for the network 130 may be connected by a network, e.g., a wireless network, a wired network, and/or an arbitrary combination of the wireless network and the wired network. The network may include the local area network, an internet, a telecommunication network, an internet of things based on the internet and/or the telecommunication network, and/or an arbitrary combination of the networks above and the like. The wired network, for example, may communicate in modes of twisted-pair, coaxial cable or optical fiber transmission and the like, and the wireless network, for example, may adopt communication modes of a 3G/4G/5G mobile communication network, Bluetooth, Zigbee or Wi-Fi and the like.

Wherein, a connection path of the network 130 may be a direct connection or an indirect connection. For example, the direct connection may include that the measuring device 110 and the client 120 implement point-to-point connection by Bluetooth, Wi-Fi (an Ad-Hoc mode) and the like. For example, the indirect connection may include that Zigbee transition connection is implemented on the basis of a PAN coordinating point and Wi-Fi transition is implemented on the basis of a Wi-Fi AP or Route.

The server 140 may be a separate server, or a server group, and each server in the group is connected by the wired or wireless network. One server group may be centralized, and for example, be a data center. The server 140 may be local, or remote. In some embodiments, the server 140 may be used for acquiring other data required in the process of correcting the measured data, as proposed by the present disclosure, e.g., a current date, time, a weather condition and the like.

The database 150 may generally refer to a device with a storage function. The database 150 is mainly used for storing data received from the client 120 and various data utilized, generated and output in the working process of the server 140. The database 150 may be local, or remote. The database 150 may be stored in various memories, e.g., a Random Access Memory (RAM), a Read Only Memory (ROM), a hard disk drive, a solid state drive, a flash memory and the like. The above-mentioned storage devices merely are illustrated as some examples, and the storage devices which can be used by the system are not limited thereto.

In some embodiments, the database 150 may store a correcting factor(s) for correcting the measured data, an additional attribute(s) of the measured data corresponding to the correcting factor, and a correcting parameter(s) of the correcting factor corresponding to the additional attribute. In some embodiments, the database 150 may be an independent device. In other embodiments, the database 150 may also be integrated in at least one of the client 120 and the server 140. For example, the database 150 may be arranged on the client 120, or may be arranged on the server 140. For another example, the database 150 may also be distributed, one portion of the database 150 is arranged on the client 120, and the other portion of the database 150 is arranged on the server 140.

The database 150 may be connected or communicated with the server 140 via the network 130, or directly connected or communicated with the server 140 or one portion thereof. A combination of the above mentioned two modes may also be adopted.

A process of a method for correcting measured data, as provided by the present disclosure, will be illustrated in detail below.

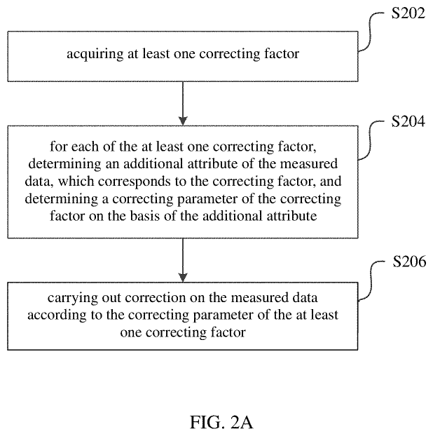

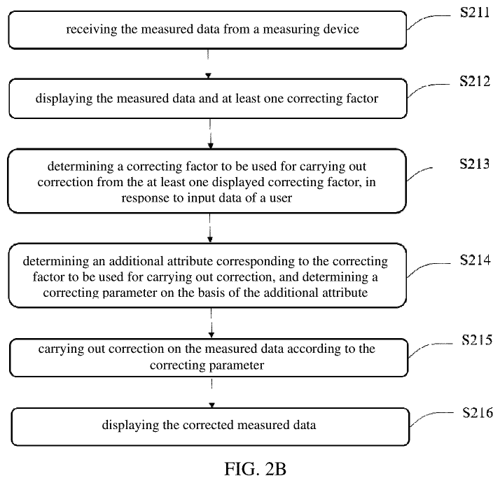

FIG. 2A shows a schematic flow chart of a method for correcting measured data according to an embodiment of the present disclosure. The method for correcting the measured data, as shown in FIG. 2A, can be implemented by utilizing the client 120 shown in FIG. 1.

As shown in FIG. 2A, in the step S202, at least one correcting factor is acquired. The correcting factor may be various factors influencing accuracy of the measured data.

For example, when a weight of a human body is measured, if the measured human body wears many clothing, wears heavy accessories or has just eaten foods, measured data acquired by the weight scale may have a deviation from an actual weight of the human body. For another example, when the laser distance meter is utilized to carry out measurement, due to limitation of a measuring environment, measured data acquired by the distance meter may include a size of the distance meter itself, and thus has a deviation from an actual distance. For yet another example, when the second chronograph is utilized to carry out timekeeping and measure time data, a user operating the second chronograph requires a certain response time, and thus, measured data acquired by the second chronograph has a deviation from actual time data.

The specific method for correcting the measured data on the basis of the correcting factor, as provided by the present disclosure, will be described below by taking a case that the measured data is weight data of the user as an example.

In some embodiments, there may be a plurality of preset correcting factors. Acquiring at least one correcting factor may refer to selecting at least one from the plurality of preset correcting factors. For example, in response to a user input, at least one of the plurality of predefined correcting factors can be selected as a correcting factor of the measured data, and used for the subsequent correcting process. In some examples, the user may input his own choice by a graphical user interface provided by an application and determine the correcting factor for the measured data.

Then, as shown in FIG. 2A, in the step S204, for each correcting factor in the at least one correcting factor, an additional attribute of the measured data, which corresponds to the correcting factor, can be determined and the correcting parameter of the correcting factor is determined on the basis of the additional attribute. The correcting parameter is used for indicating an influence degree of the correcting factor for the measured data. As mentioned above, the correcting factor may be various factors influencing accuracy of the measured data. It should be understood that different correcting factors may influence accuracy of the measured data in different modes to varying degrees. Moreover, even for the same correcting factor, there may be a plurality of variables influencing the measured data. Therefore, according to the acquired correcting factor, at least one additional attribute which corresponds to the correcting factor and influences accuracy of the measured data can be determined. The additional attribute can be used for determining the correcting parameter of the correcting factor. The correcting parameter can indicate the influence degree of the correcting factor for the measured data, i.e., a deviation degree of the measured data from real data.

According to the embodiments of the present disclosure, the additional attribute may be at least one of user input information and pre-acquired statistical information. For example, for each correcting factor, statistics can be carried out according to a result of a questionnaire survey so as to determine the deviation degree of the measured data from the real data, which is caused by the additional attribute associated with the correcting factor, i.e., the correcting parameter of the correcting factor determined on the basis of the additional attribute. In some embodiments, the correcting parameter for each correcting factor can be updated by the regular questionnaire surveys or by regularly collected related information input by the user. A statistical result of the questionnaire surveys can be pre-stored in a database. In the process of correcting the measured data, the stored statistical result can be acquired by accessing the database.

In some embodiments, the correcting parameter corresponding to each additional attribute can be pre-stored in the database in a manner of associating with the additional attribute in advance. Therefore, in the process of correcting the measured data, the correcting parameter associated with the additional attribute can be searched from the database according to the additional attribute so as to implement determination on the correcting parameter of the correcting factor on the basis of the additional attribute.

In some embodiments, the correcting parameter may be represented as an absolute value of deviation of the measured data from the real data. In other embodiments, the correcting parameter may be represented as a percentage of deviation of the measured data from the real data.

According to the embodiments of the present disclosure, in a case that the measured data is the weight data of the user, the at least one correcting factor includes one or more of a wearing condition, a handheld condition and a dietary condition.

By taking the case that the measured data is the weight data of the user as an example, the correcting factor may be the wearing condition. The wearing condition may be used for representing influence of clothing, a watch, a jewelry and the like worn by the user on the weight data. The additional attribute of the measured data, which corresponds to the wearing condition, can include at least one of date information, a weather condition, a geographic position and physiological data of the user.

The method provided by the present disclosure will be described below by taking a case that the wearing condition is clothing worn by the user as an example. For example, clothing worn by the user may include clothes, shoes, a scarf, a hat, gloves and the like.

For a case that the additional attribute includes date information, the date information in the measuring process can be acquired by a network time server or by reading system time of an electronic device used as the client, and the date information is used as the additional attribute. A season when weight measurement is carried out on the user can be determined by utilizing the date information in the measuring process, so that a weight of the clothing worn by the user can be determined according to season information. For example, a weight of clothing worn by the user in winter is greater than a weight of clothing worn by the user in summer. Weights of clothing worn by the user in different seasons can be acquired by pre-surveying wearing conditions of people in different regions. For example, a questionnaire survey can be made to the user so as to acquire thicknesses and numbers of clothes, types of shoes (e.g., slippers or boots) and the like, which are worn by the user in different seasons. In some examples, the weights of clothing worn by the user in different seasons can be determined by carrying out statistics on the result of the questionnaire survey carried out in advance. For example, an average value of the weights of clothing worn in different seasons by the user participating in the questionnaire survey can be used as the correcting parameter for the wearing condition in different seasons. The process above can be represented as a process of determining the additional attribute, i.e., the date information, associated with the wearing condition, by the wearing condition used as the correcting factor so as to determine the correcting parameter according to the date information.

For a case that the additional attribute includes geographic information, the geographic position of the user can be acquired in a manner of positioning by a satellite positioning system (GPS, a Beidou satellite and the like) or cellphone base stations so as to respond to changes of wearing habits and the like of the users in different regions. For example, in northeast China and northern China, even in the autumn and winter environments with the same cold degrees, wearing habits and types of clothes are also different (difference of mink cashmere type products and cotton-padded clothes), and this case can assist in correcting the weight difference caused by the wearing habits and the like of the users in different regions.

For a case that the additional attribute includes date information and geographic information, a weather condition of a corresponding place, such as a temperature, a wind direction, wind strength and the like, can be acquired from, for example, a weather server according to the geographic information and the date information. For example, the weather condition when the weight data is acquired can be determined in connection with geographic information of a position where the user is positioned when the weight is measured and the date information. The weight of clothing worn by the user can be more accurately determined in connection with the specific weather condition. For example, the user wears more clothing in a case of bad weather (for example, high wind) than in a case of good weather, and thus, the weight is also greater. Similarly, an average value of the weights of clothing worn in different weather cases by the user participating in the questionnaire survey can also be used as the correcting parameter for the wearing condition in different weather cases by the result of the questionnaire survey carried out in advance.

For a case that the additional attribute includes physiological data, the physiological data of the user can be acquired according to information input by the user or user information pre-stored in the database. For example, when the user uses an application for health on the client, the user may be required to input basic information of the user in his first use, such as age, height, gender, body shape and the like. As an example, the body shape may be represented as an initial weight value of the user.

The physiological data of the user may include one or more of height data, body shape data and gender data of the user. For example, a size of clothing worn by the user can be determined according to the height, body shape and gender of the user, and the weight of the worn clothing can be determined according to the determined size. It should be understood that the larger the size of the clothing is, the greater the weight of the clothing is. Therefore, the users participating in the survey are divided into different categories according to height, body shape and gender, and the weights of clothing worn by the users belonging to different categories can be respectively subjected to statistics. In this case, a user category to which the measured user belongs can be determined according to the physiological data of the measured user, and an average value of weights of clothing worn by users in this category is used as the correcting parameter for the wearing condition.

In some embodiments, an influence degree of the clothing worn by the user on the measured weight data can be determined according to at least one of the date information, the weather condition, the geographic position and the physiological data of the user. For example, in a case that the acquired correcting factor is the wearing condition, the additional attribute of the weight data, which corresponds to the wearing condition, can be determined as the date information and the gender of the user. As an example, in a case of determining that a current date is Mar. 25, 2019, the season is early spring, the position is Beijing and the user is a female, it can be searched from the database that under this weather condition, an average female clothing weight in Beijing is 450 g. If the additional attribute further includes the weather condition, in a case of determining that on that day, there is moderate rain and the minimum temperature is 8° C., the clothing weight can be determined as 700 g.

In some embodiments, the wearing condition may further include the watch worn by the user. In this case, the additional attribute corresponding to the worn watch may include the gender of the user and device equipment of a client device.

For example, an average weight of watches worn by males and an average weight of watches worn by females can be subjected to statistics in advance and used as the correcting parameter of the watch.

For another example, the watch worn by the user can be determined according to the device information of the client. For example, if a brand of an electronic device is determined according to device equipment, it can be considered that the watch worn by the user is a product of the same brand, so that a weight of the product of the same brand can be determined as the correcting parameter. For example, if the client device is an iPhone, a weight of the Apple watch series 4 with the specification of 40 mm can be determined as the correcting parameter and is about 30.1 g.

For yet another example, it can be determined whether there is information about a watch connected to the client device by accessing system information of the client. If information about the watch connected to the client device exists, device information of the connected watched can be determined according to the system information so as to determine a model of the watch worn by the user. Thus, the weight the watch of the determined model is determined as the correcting parameter.

In some embodiments, the wearing condition may further be the jewelry worn by the user. In this case, the additional attribute corresponding to the worn jewelry may be at least one of the gender data of the user and historical shopping information of the user.

In some examples, an average weight of jewelry worn by males and an average weight of jewelry worn by females can be subjected to statistics in advance and used as the correcting parameter.

In other examples, a jewelry wearing condition of the user can be determined by accessing the historical shopping information of the user, which is stored in the database. For example, if the historical shopping information of the user indicates that the user often buys jewelry (for example, a total shopping quantity exceeds a predefined threshold or a shopping frequency exceeds a predefined threshold), it can be considered that the user likes to wear jewelry, and the correcting parameter corresponding to the jewelry wearing condition can be correspondingly added. If the historical shopping information of the user indicates that the user seldom buys jewelry (for example, the total shopping quantity is smaller than the predefined threshold or the shopping frequency is smaller than the predefined threshold), it can be considered that the user seldom wears jewelry, and the correcting parameter corresponding to the jewelry wearing condition can be correspondingly reduced.

According to the embodiments of the present disclosure, when the correcting factor is the handheld condition, the additional attribute of the measured data, which corresponds to the handheld condition, includes device information of a handheld device for receiving the measured data. In some embodiments, the user may utilize a smartphone or other handheld electronic devices used as the client to record a measuring result. Therefore, when the weight scale or the body fat scale is used for carrying out measurement, the user needs to hold the handheld electronic device for recording the measuring result with a hand in the measuring process.

For example, the device information of the handheld device can be determined by reading the system information of the electronic device so as to determine a model of the handheld device. In this case, a weight of the device can be determined by the model of the handheld device. For example, when it is determined that the model of the client device is iPhone Xs, it can be searched from the database that a weight of the determined model of device is 177 g, and the determined device weight is used as the correcting parameter of the handheld condition.

Moreover, the correcting factor may further include the dietary condition of the user. In some embodiments, when the weight data is measured, if the user has just eaten a meal, the measuring result may be greater than an actual weight of the user, and thus, inaccuracy of the weight data, which is caused by diet of the user, needs to be corrected.

In some embodiments, when the correcting factor is the dietary condition, the additional attribute of the measured data, which corresponds to the dietary condition, includes at least one of time information, the geographic position and the gender information of the user.

The time information when the weight is measured, e.g., time information represented with a 24-hour system or a 12-hour system, can be acquired by a network time server or by reading system time of the electronic device used as the client. According to the time information, whether the user has eaten foods at the moment when the weight is measured and a weight of the eaten foods can be determined. For example, when it is determined that time when the weight is measured is half past twenty, the geographic position is Beijing and the user is a female, according to a statistical result pre-stored in the database, it can be determined that at the moment, the user has eaten dinner, and thus, an average dinner food intake of females can be determined as the correcting parameter of the dietary condition. For example, the average dinner food intake of females may be 400 g.

In some embodiments, historical dietary information of the user may be recorded by an application in the client, and according to historical mealtime and a historical dietary food intake of the user, the correcting parameter of the dietary condition, which is used for the user, is determined. For example, the user can record his own dietary information in a manner of taking photos and uploading the photos. The server can carry out image identification on the photos uploaded by the user so as to determine the historical dietary information of the user, i.e., a food intake mass before the weight is measured.

Then, as shown in FIG. 2A, in the step S206, the measured data can be corrected according to the correcting parameter of the at least one correcting factor. For example, the influence degree of each correcting factor on the measured data can be determined by a result output in the step S204, and the measured data can be corrected by the correcting parameter output in the step S204. In some embodiments, the correcting parameter of the correcting factor can be determined by at least one correcting factor and on the basis of the method above, so as to carry out correction on the measured data by utilizing the correcting parameter. In other embodiments, after the correcting factor is selected and the correcting parameter of the correcting factor is utilized to carry out correction, it may also be selected to eliminate influence of the correcting factor on the measured data. Namely, the corrected measured data is recovered into the data before correction.

Although the principle of the present disclosure has been described above by taking the case that the measured data is the weight data as the example, those skilled in the art should understand that when the measured data is the distance data or the time data, those skilled in the art can set the correcting factors for the distance data or the time data and the associated additional attributes according to actual situations so as to implement correction on the measured data.

For example, when the measured data is distance data, the correcting factor may be a measuring device condition. In some practical applications, a length of the measuring device cannot be subtracted from the distance data obtained by the measuring device, resulting in a deviation between the measured distance data and an actual distance data. According to the method provided by the present disclosure, the device information of the measuring device can be set as the additional attribute. Therefore, according to the device information of the measuring device, the length of the measuring device can be determined as the correcting parameter, and the measured data is corrected by utilizing the length of the measuring device.

For another example, when the measured data is time data, the correcting factor may be a response speed of the user. At the moment, the additional attribute for the response time can be set as age information. For example, when the user is too old or too young, it can be considered that the response speed of the user is low. Therefore, response time of users in different age groups can be predetermined, and the response time of the users in this age group is determined as the correcting parameter of the time data of the user according to the age of the user.

By utilizing the technical solution provided by the present disclosure, the additional attribute of the measured data, which corresponds to the correcting factor, can be determined according to the correcting factor influencing the measured data, and thus, the correcting parameter of the correcting factor can be determined on the basis of the determined additional attribute so as to carry out correction on the measured data on the basis of the correcting parameter. Therefore, by presetting an association relationship between the additional attribute and the correcting factor and the influence degree of each additional attribute on the measured data, the measured data can be more conveniently, rapidly and accurately corrected so as to eliminate influence of each correcting factor on the measured data. Compared to the data before correction, the corrected measured data has higher accuracy, which is beneficial to subsequent application carried out on the basis of the measured data, and for example, for the case that the measured data is the weight data, the accurate weight data is more beneficial to monitoring of the user on the weight and particularly users who are doing weight management.

According to another aspect of the present disclosure, further provided is an interactive method for correcting measured data, and FIG. 2B shows a schematic flow chart of the interactive method for correcting the measured data according to an embodiment of the present disclosure.

As shown in FIG. 2B, in the step S211, the measured data from the measuring device is received; in the step S212, the measured data and at least one correcting factor are displayed; in the step S213, in response to input data of the user, the correcting factor to be used for carrying out correction is determined from the at least one displayed correcting factor; in the step S214, the additional attribute corresponding to the correcting factor to be used for carrying out correction is determined, and the correcting parameter is determined on the basis of the additional attribute; in the step S215, the measured data is corrected according to the correcting parameter; and in the step S216, the corrected measured data is displayed.

In some embodiments, the displaying at least one correcting factor is determined on the basis of a priority of the correcting factor.

In some embodiments, the displaying at least one correcting factor includes: displaying the at least one correcting factor in a form of an icon or a list.

In some embodiments, the interactive method further includes: after displaying the corrected measured data, changing a display mode of the at least one correcting factor; and in response to the user canceling the input data, updating display of the corrected measured data.

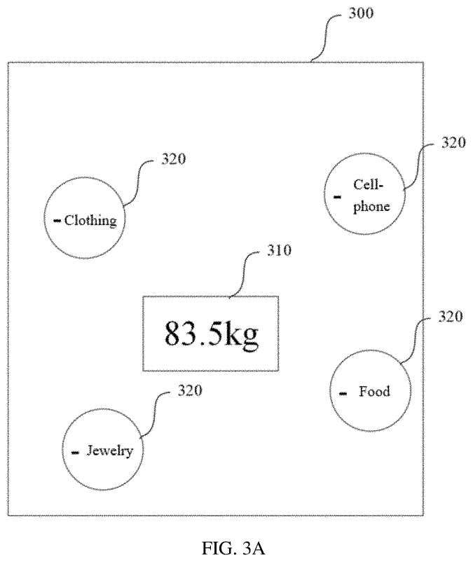

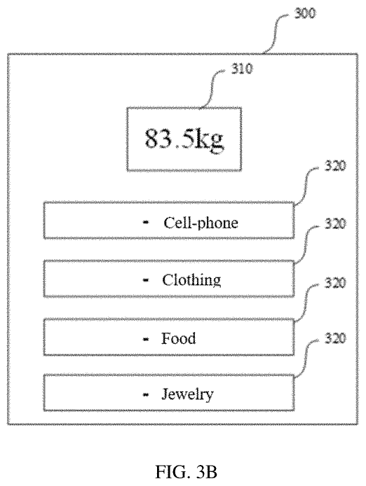

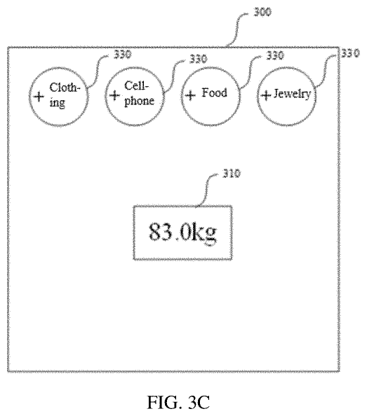

FIG. 3A shows an exemplary schematic diagram of a graphical user interface according to an embodiment of the present disclosure. The interactive method for correcting the measured data, as provided by the embodiment of the present disclosure, will be described in detail below in connection with FIG. 3A.

In an example shown in FIG. 3A, the measuring device may be a smart weight scale or a smart body fat scale, and the client may be a smartphone or a tablet personal computer, wherein the measuring device can send the measured data to the client via a network. The interactive method for correcting the measured data, as shown in FIG. 2B, can be implemented by utilizing an application installed on the smartphone.

As shown in FIG. 3A, when the client receives the measured data, an application for correcting the measured data (also referred to as a correcting program) can be started up, and a graphical user interface 300 is displayed to a user. An icon 310 indicates the measured data. In the example shown in FIG. 3A, the measured data is 83.5 kg. In the correcting program, a plurality of different correcting factors (for example, an icon 320) can be displayed to the user in the graphical user interface 300. The user can select one or more of the correcting factors according to an actual situation under which the data correction is carried out. For example, the user can select an icon “-cellphone” (which represents a handheld condition) so as to eliminate influence of a case that the user holds a cellphone with a hand on a measuring result in the weight measuring process. Correspondingly, the user can also select one or more from icons “-clothing” (which represents a wearing condition for clothing), “-food” (which represents a dietary condition) and “-jewelry” (which represents a wearing condition for jewelry) so as to respectively eliminate influence of the correcting factors represented with those icons on the measuring result. Measurement on weight data can be implemented herein by utilizing the steps of correcting the measured data, as shown in FIG. 2A, and is not repeated herein.

In some embodiments, display information of each correcting factor in the graphical user interface 300 may be determined according to the priority of the correcting factor. For example, the priority of each correcting factor can be determined according to historical use information of the user. The higher a use frequency is, the higher the priority of the correcting factor is. For example, during the last 20 times of weight measurement, the user selects “-cellphone” for 18 times, selects “-clothing” for 12 times, selects “-food” for 8 times and selects “-jewelry” for once, so it can be considered that the correcting factor indicated by “-cellphone” has the highest priority, the correcting factor indicated by “-clothing” has the second priority, the correcting factor indicated by “-food” has the third priority, and the correcting factor indicated by “-jewelry” has the lowest priority.

According to the priority of the correcting factor, which is determined by the method as mentioned above, the display information of the icon indicating the correcting factor can be determined according to the priority so as to achieve an effect that the higher the priority is, the more easily the user operates and selects.

For example, as shown in FIG. 3A, the icon “-cellphone” of the correcting factor with the highest priority can be placed on the top right corner of the graphical user interface, the icon “-clothing” of the correcting factor with the second priority can be placed on the top left corner of the graphical user interface, the icon “-food” of the correcting factor with the third priority can be placed on the bottom right corner of the graphical user interface, and the icon “-jewelry” of the correcting factor with the lowest priority can be placed on the bottom left corner of the graphical user interface. Therefore, a higher priority of a correcting factor indicates that an icon of the correcting factor is placed at a position where the user operates more easily.

FIG. 3B shows another example of setting the display information of the correcting factor according to the priority. As shown in FIG. 3B, each correcting factor can be displayed in the form of a list, and a display sequence of each correcting factor in the list can be determined according to the priority.

FIG. 3C shows yet another exemplary schematic diagram of a graphical user interface according to an embodiment of the present disclosure. As shown in FIG. 3C, after the user respectively selects “-cellphone”, “-clothing”, “-food” and “-jewelry” to carry out correction on the weight data, the weight data in a measured data box is changed into 83.0 kg from 83.5 kg in FIG. 3A, and display positions of the icons indicating the correcting factors are changed to be suspended at the upper part of the graphical user interface. If the user expects to cancel the selected correcting factor, the user can select an icon 330 shown in FIG. 3C so as to cancel a correcting effect of the correcting factor.

By utilizing the embodiments provided by the present disclosure, it can be implemented that the user conveniently corrects the result of the measured data, and randomly selects or cancels the correcting result of one or more correcting factors on the measured data according to the actual situations.

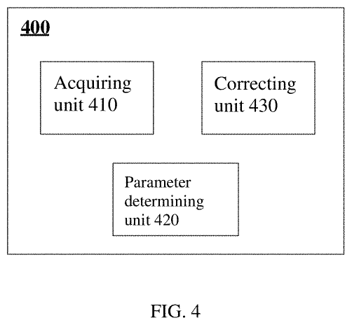

FIG. 4 shows a schematic diagram of an apparatus for correcting measured data according to an embodiment of the present disclosure. As shown in FIG. 4, the apparatus 400 for correcting the measured data may include an acquiring unit 410, a parameter determining unit 420 and a correcting unit 430. In some embodiments, the client 120 shown in FIG. 1 can be implemented by utilizing the apparatus 400 for correcting the measured data, as shown in FIG. 4.

The acquiring unit 410 may be configured to acquire at least one correcting factor. The correcting factor is various factors influencing accuracy of the measured data.

A specific method for carrying out correction by utilizing the correcting factor will be described below by taking a case that the measured data is weight data of a user as an example.

When the measured data is the weight data of the user, the at least one correcting factor includes one or more of a wearing condition, a handheld condition and a dietary condition.

In some embodiments, there may be a plurality of preset correcting factors. Acquiring at least one correcting factor may refer to selecting at least one from a plurality of preset correcting factors. For example, in response to a user input, at least one can be selected as a correcting factor of the measured data from a plurality of predefined correcting factors, and used for the subsequent correcting process. In some examples, the user may input own choice by a graphical user interface provided by an application and determine the correcting factor for the measured data.

The parameter determining unit 420 may be configured, for each of the at least one correcting factor, determine an additional attribute of the measured data, which corresponds to the correcting factor, and determine a correcting parameter of the correcting factor on the basis of the determined additional attribute.

The additional attribute may be user input information and/or pre-acquired statistical information. For example, for each correcting factor, a deviation degree of the measured data from real data, which is caused by the additional attribute associated with the correcting factor, can be determined according to a result of a questionnaire survey. In some embodiments, the correcting parameter for each correcting factor can be updated by the regular questionnaire survey or by regularly collecting related information input by the user. A statistical result of the questionnaire survey can be pre-stored in a database. In the process of correcting the measured data, the stored statistical result can be acquired by accessing the database.

In some embodiments, the correcting parameter corresponding to each additional attribute can be pre-stored in the database in a mode of associating with the additional attribute. Therefore, in the process of correcting the measured data, the correcting parameter associated with the additional attribute can be searched from the database according to the additional attribute.

In some embodiments, the correcting parameter may be represented as an absolute value of deviation of the measured data from the real data. In other embodiments, the correcting parameter may be represented as a percentage of deviation of the measured data from the real data

By taking the case that the measured data is the weight data of the user as an example, when the correcting factor is the wearing condition, the additional attribute of the measured data, which corresponds to the wearing condition, may include at least one of date information, a weather condition, a geographic position and physiological data of the user, wherein the wearing condition may be used for representing influence of clothing, a watch and an accessories such as jewelry and the like, which are worn by the user, on the weight data.

The date information when the weight is measured can be acquired by a network time server or by reading system time of an electronic device used as the client. A season when the user is subjected to weight measurement can be determined by utilizing a date when measurement is carried out, so that a weight of the clothing worn by the user can be determined according to season information.

A weather condition of a corresponding place, such as a temperature, a wind direction, wind strength and the like, can be acquired from a weather server according to the geographic information and the date. For example, the weather condition when the weight data is acquired can be determined in connection with the geographic information of the user and the date information when the weight is measured. The weight of the clothing worn by the user can be more accurately determined in connection with the specific weather condition.

The physiological data of the user may include one or more of height data, body shape data and gender data of the user. For example, a size of the clothing worn by the user can be determined according to the height, body shape and gender of the user, and the weight of the worn clothing can be determined according to the determined size.

In some embodiments, the wearing condition may further include a watch worn by the user. In this case, the additional attribute corresponding to the worn watch may include the gender of the user and device equipment of a client device.

For example, an average weight of watches worn by males and an average weight of watches worn by females can be subjected to statistics in advance and used as the correcting parameter of the watch. For another example, the watch worn by the user can be determined according to the device information of the client. For yet another example, it can be judged whether there is watch information connected to the client device by accessing system information of the client. If there is the watch information connected to the client device, device information of the connected watched can be determined according to the system information so as to determine a model of the watch worn by the user, thereby determining the weight of the model of watch as the correcting parameter.

In some embodiments, the wearing condition may further be the jewelry worn by the user. In this case, the additional attribute corresponding to the worn jewelry may be at least one of the gender data of the user and historical shopping information of the user.

In some examples, an average weight of jewelry worn by males and an average weight of jewelry worn by females can be subjected to statistics in advance and used as the correcting parameter.

In other examples, a jewelry wearing condition of the user can be determined by accessing the historical shopping information of the user, which is stored in the database.

When the correcting factor is the handheld condition, the additional attribute of the measured data, which corresponds to the handheld condition, includes device information of a handheld device for receiving the measured data. In some embodiments, the user may utilize a smartphone or other handheld electronic devices used as the client to record a measuring result.

For example, the device information of the handheld device can be determined by reading the system information of the electronic device so as to determine a model of the handheld device. In this case, a weight of the device can be determined by the model of the handheld device.

Therefore, the correcting factor may further include the dietary condition of the user. In some embodiments, when the weight is measured, if the user has just eaten a meal, the measuring result may be greater than an actual weight of the user, and thus, inaccuracy of the weight data, which is caused by diet of the user, needs to be corrected.

In some embodiments, when the correcting factor is the dietary condition, the additional attribute of the measured data, which corresponds to the dietary condition, includes at least one of time information, the geographic position and the gender information of the user.

The time information in the measuring process, e.g., time information represented with a 24-hour system or a 12-hour system, can be acquired by the network time server or by reading system time of the electronic device used as the client. According to the time information, whether the user has eaten foods at the moment when the weight is measured and a weight of the eaten foods can be judged.

In some embodiments, historical dietary information of the user may be recorded by an application in the client, and according to historical mealtime and a historical dietary food intake of the user, the correcting parameter of the dietary condition, which is used for the user, is determined.

The correcting unit 430 may be configured to carry out correction on the measured data according to the correcting parameter of the at least one correcting factor. For example, an influence degree of each correcting factor on the measured data can be determined by a result output by the parameter determining unit 420, and the measured data can be corrected by the correcting parameter output by the parameter determining unit 420. In some embodiments, the correcting parameter of the correcting factor can be determined by at least one correcting factor and on the basis of the method above, so as to carry out correction on the measured data by utilizing the correcting parameter. In other embodiments, after the correcting factor is selected and the correcting parameter of the correcting factor is utilized to carry out correction, it may also be selected to eliminate influence of the correcting factor on the measured data. Namely, the corrected measured data is recovered into the data before correction.

According to some embodiments of the present disclosure, the apparatus 400 may further include a display unit (not shown in FIG. 4). The display unit may be configured to: determine display information of an icon indicating each correcting factor according to priority information of the at least one correcting factor. A specific implementing process of the display unit can refer to the steps described above in connection with FIG. 3A to FIG. 3C, and is not repeated herein.

Although the principle of the present disclosure has been described above by taking the case that the measured data is the weight data as the example, those skilled in the art should understand that when the measured data is the distance data or the time data, those skilled in the art can set the correcting factors for the distance data or the time data and the associated additional attributes according to the actual situations so as to implement correction on the measured data.

By utilizing the technical solution provided by the present disclosure, the additional attribute of the measured data, which corresponds to the correcting factor, can be determined according to the correcting factor influencing the measured data, and thus, the correcting parameter of the correcting factor can be determined on the basis of the determined additional attribute so as to carry out correction on the measured data. Therefore, by presetting an association relationship between the additional attribute and the correcting factor and the influence degree of each additional attribute on the measured data, the measured data can be more conveniently corrected so as to eliminate influence of each correcting factor on the measured data.

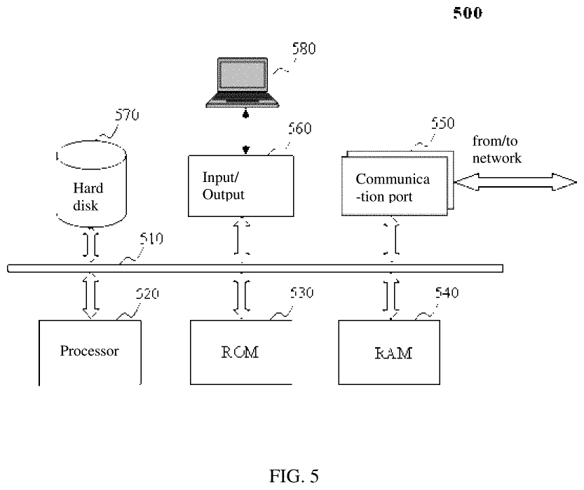

Moreover, the method or the apparatus according to the embodiments of the present disclosure may also be implemented by means of a framework of a computing device as shown in FIG. 5. FIG. 5 shows the framework of the computing device. As shown in FIG. 5, the computing device 500 may include a bus 510, one or more processors 520, an ROM 530, an RAM 540, a communication port 550 connected to a network, an input/output component 560, a hard disk 570 and the like. A storage device in the computing device 500, e.g., the ROM 530 or the hard disk 570, can store various data or files used by the method and interactive method for correcting the measured data, as provided by the present disclosure, and a program instruction executed by a Central Processing Unit (CPU). The computing device 500 may further include a user interface 580. Certainly, the framework shown in FIG. 5 is just exemplary, and when different devices are implemented, one or more components in the computing device shown in FIG. 5 can be omitted according to actual demands.

The embodiments of the present disclosure may also be implemented as a computer readable storage medium. The computer readable storage medium according to the embodiments of the present disclosure stores a computer readable instruction. When the computer readable instruction is operated by a processor, the method according to the embodiments of the present disclosure, as described with reference to the drawings above, can be executed. The computer readable storage medium includes, but is not limited to, for example, a volatile memory and/or a nonvolatile memory. The volatile memory, for example, may include an RAM and/or a cache and the like. The nonvolatile memory for example may include an ROM, a hard disk, a flash memory and the like.

In the embodiments of the present disclosure, the processor may be a logical operating device with data processing capacity and/or program executing capacity, such as a CPU, a Field Programmable Gate Array (FPGA), a Micro Control Unit (MCU), a Digital Signal Processor (DSP), an Application-Specific Integrated Circuit (ASIC) and the like.

According to another aspect of the present disclosure, further provided is an electronic device for correcting measured data. The device includes a memory and a processor, wherein an instruction is stored in the memory, and when the instruction is executed by utilizing the processor, the processor executes the above-mentioned method for correcting the measured data, or executes the above-mentioned interactive method for correcting the measured data.

Based on the embodiments above, the present disclosure further provides a measuring device, including the electronic device for correcting the measured data, which implements the method in the embodiments above; or being communicatively connected with the electronic device for correcting the measured data, which implements the method in the embodiments above.

For example, the measuring device is a weight or body fat measuring apparatus, and the electronic device for correcting the measured data is a cellphone or a tablet personal computer.

For example, the weight or body fat measuring apparatus may be communicatively connected with the cellphone or the tablet personal computer in a wireless connecting mode such as Bluetooth and Wi-Fi.

Those skilled in the art should understand that various modifications and improvements can be made to the contents disclosed by the present disclosure. For example, various devices or components described above may be implemented by hardware, or by software, firmware or a combination of some or all of the hardware, software and firmware.

In addition, as shown in the present disclosure and claims, unless exceptional cases are definitely indicated in the context, words such as “one”, “a/an”, “a/an” and/or “the” or the like do not particularly denote there is only one, but rather indicate there are more. Generally speaking, terms such as “include” and “comprise” just denote that steps and elements which have been definitely marked are included, but those steps and elements do not constitute an exclusive enumeration, the method and the device may also include other steps or elements.

In addition, although the present disclosure makes various references to certain units in the system according to the embodiments of the present disclosure, any numbers of different units can be used and operated on the client and/or the server. The unit merely is illustrative, and different aspects of the system and the method may use different units.

In addition, the flow chart is used in the present disclosure for illustrating the operations executed by the system according to the embodiments of the present disclosure. It should be understood that the previous or subsequent operations are not necessarily accurately executed in sequence. On the contrary, various steps may be processed according to an inverted sequence or simultaneously. Meanwhile, other operations may also be added into those processes, or a certain step or some steps of operations may be removed from those processes.

Unless otherwise defined, all the terms (including the technical and scientific terms) here should be of general meaning as understood by those ordinarily skilled in the art. It also should be understood that those terms defined in ordinary dictionaries for example should be explained as meanings consistent with their meanings in the context of the related art, rather than explained as idealized or highly formalized meanings, unless expressly defined.

The above is only used for illustrating the present disclosure, but should not be understood as limitation thereto. Although some exemplary embodiments of the present disclosure are described, those skilled in the art will easily understand that many modifications can be made to the exemplary embodiments without departure from the novel teaching and advantages of the present disclosure. Therefore, all the modifications intend to fall within the scope of the present disclosure, as defined by the claims. It should be understood that the above is used for illustrating the present disclosure, but should not be understood as limitation to the disclosed specific embodiments, and modifications to the disclosed embodiments and other embodiments intend to fall within the scope of the appended claims. The present disclosure is defined by the claims and equivalents thereof.

1. A method for correcting measured data, comprising:
acquiring at least one correcting factor;
for each correcting factor of the at least one correcting factor,
determining an additional attribute of the measured data corresponding to the correcting factor, and
determining a correcting parameter of the correcting factor on the basis of the additional attribute; and
carrying out correction on the measured data according to the correcting parameter of the at least one correcting factor.

2. The method according to claim 1, wherein the additional attribute is at least one of user input information and pre-acquired statistical information.

3. The method according to claim 1, wherein the measured data is weight data of a user, and the at least one correcting factor includes at least one of a wearing condition, a handheld condition and a dietary condition.

4. The method according to claim 3, wherein when the correcting factor is the wearing condition, the additional attribute of the measured data corresponding to the wearing condition includes at least one of date information, a weather condition, a geographic position and physiological data of the user.

5. The method according to claim 4, wherein the physiological data of the user includes at least one of height data, body shape data and gender data of the user.

6. The method according to claim 3, wherein when the correcting factor is the handheld condition, the additional attribute of the measured data corresponding to the handheld condition includes device information of a handheld device for receiving the measured data.

7. The method according to claim 3, wherein when the correcting factor is the dietary condition, the additional attribute of the measured data corresponding to the dietary condition includes at least one of time information, geographic position and gender information of the user.

8. The method according to claim 1, wherein acquiring at least one correcting factor includes: in response to a user input, selecting at least one from a plurality of predefined correcting factors as the correcting factor of the measured data.

9. The method according to claim 8, further comprising:
determining display information of an icon for indicating each correcting factor according to priority information of the at least one correcting factor.

10. An interactive method for correcting measured data, comprising:
receiving the measured data from a measuring device;
displaying the measured data and at least one correcting factor;
determining a correcting factor to be used for carrying out correction from at least one displayed correcting factor, in response to input data of a user;
determining an additional attribute corresponding to the correcting factor to be used for carrying out correction, and determining a correcting parameter on the basis of the additional attribute;
carrying out correction on the measured data according to the correcting parameter; and
displaying the corrected measured data.

11. The interactive method according to claim 10, wherein the displaying at least one correcting factor is determined on the basis of a priority of the correcting factor.

12. The interactive method according to claim 10, wherein the displaying at least one correcting factor includes: displaying the at least one correcting factor in a form of an icon or a list.

13. The interactive method according to claim 12, further comprising:
changing a display mode of the at least one correcting factor, after displaying the corrected measured data; and
updating display of the corrected measured data, in response to the user canceling the input data.

14. An apparatus for correcting measured data, comprising:
an acquiring unit, configured to acquire at least one correcting factor;
a parameter determining unit, configured to, for each correcting factor in the at least one correcting factor, determine an additional attribute of the measured data corresponding to the correcting factor and determine a correcting parameter of the correcting factor on the basis of the additional attribute; and
a correcting unit, configured to correct the measured data according to the correcting parameter of the at least one correcting factor.

15. The apparatus according to claim 14, wherein the additional attribute is at least one of user input information and pre-acquired statistical information.

16. The apparatus according to claim 14, wherein the measured data is weight data of a user, and the at least one correcting factor includes at least one of a wearing condition, a handheld condition and a dietary condition.

17. An electronic device for correcting measured data, comprising a memory and a processor, wherein instructions are stored in the memory, and when the instructions are executed by utilizing the processor, the processor executes the method for correcting the measured data according to claim 1.

18. A computer readable storage medium, stored thereon instructions, wherein when the instructions are executed by a processor, the processor executes the method for correcting the measured data according to claim 1.

19. A measuring device, comprising or being communicatively connected with the electronic device for correcting the measured data according to claim 17.

20. The measuring device according to claim 19, wherein the measuring device is a weight or body fat measuring apparatus, and the electronic device for correcting the measured data is a cellphone or a tablet personal computer.
